(19) United States
(12) Patent Application Publication
(10) Pub. No.: US 2024/0173356 A1
(43) Pub. Date: May 30, 2024

(54) Extracellular Vesicles From Human Induced Pluroptent Stem Cells

(71) Applicant: Florida State University Research Foundation, Inc., Tallahassee, FL (US)

(72) Inventor: Yan Li, Tallahassee, FL (US)

(21) Appl. No.: 18/524,275

(22) Filed: Nov. 30, 2023

Related U.S. Application Data
(60) Provisional application No. 63/385,526, filed on Nov. 30, 2022.

(51) Int. Cl. A61K 35/30; A61K 9/00; A61K 9/06; A61K 47/36; A61P 9/10; C12N 5/00; C12N 5/079; C12N 15/113

(52) U.S. Cl. CPC A61K 35/30; A61K 9/0019; A61K 9/06; A61K 47/36; A61P 9/10; C12N 5/0018; C12N 5/0618; C12N 15/113; C12N 2501/115

(57) ABSTRACT

Extracellular vesicles (EVs) from human induced pluripotent stem cells (hiPSCs) may be produced by forming 3-D brain organoids of the hiPSCs in a dynamic bioreactor and collecting the EVs from the bioreactor. The EVs may be used for treating ischemic conditions.

### CROSS-REFERENCE TO RELATED APPLICATION

This claims the benefit of priority from Application No. 63/385,526, filed Nov. 30, 2022, which is incorporated by reference in its entirety.

### STATEMENT REGARDING FEDERALLY SPONSORED RESEARCH OR DEVELOPMENT

This invention was made with government support under Contract No. R01NS125016 awarded by the National Institutes of Health (NIH). The government has certain rights in this invention.

### FIELD

This relates to the field of extracellular vesicles and, more particularly extracellular vesicles having therapeutic uses.

### BACKGROUND

Human induced pluripotent stem cells (hiPSCs) and human mesenchymal stem cells (hMSCs) secrete extracellular vesicles (EVs). EVs have been generated using a number of different methods, such as by static 2D cultures.

More than 4 million Americans are living with the effects of stroke, without an effective treatment to improve functional recovery. The stroke-damaged site is especially cytotoxic to neurons because of the high susceptibility to reactive oxygen species and pro-inflammatory enzymes. Stem cell therapy using adult neural progenitor cells (NPCs) is limited by insufficient cell supply while mesenchymal stem cells (MSCs) have achieved limited outcomes due to short-term survival.

Membrane-bound microvesicles are EVs that represent an active component of the cell secretome, harboring microRNA (miRNA) cargo that provides a significant contribution to secretome effects. Derivation of brain organoids from hiPSCs has emerged as a promising approach for mimicking 3-D human brain tissue.

hiPSC-derived brain organoids offer a virtually unlimited supply of human NPCs. However, current knowledge on the therapeutic benefits of EVs secreted by iPSC-derived brain organoids is limited. In particular, the influence of miRNA cargo on brain tissue repair remains unknown.

In the United States, stroke is the third leading cause of death and disability. Ischemic stroke, representing 85% of all stroke cases, occurs when blood clots block the blood vessels to the brain and cause neurological deficits leading to paralysis, disability, or death. Currently, there is no effective treatment to improve functional recovery after ischemic stroke. Thrombolytic therapy can be given to patients within 4-5 hours after stroke; however, less than 5% of US patients are administered this therapy. Medications targeting more than one pathogenic pathway during neurogenesis may represent a better therapeutic strategy. In fact, stem cell therapy has emerged as a promising treatment. NPCs are able to replace degenerated neural cells as well as secrete trophic factors to stimulate host neuron growth.

Transplantation of hiPSC-NPCs have exhibited functional recovery and electrophysiological properties of mature neurons and have proven to be a promising approach for treating stroke-damaged brain. However, cell engraftment and functional tissue integration need to be improved.

Human mesenchymal stem cells (hMSCs), instead of direct cell replacement, secrete neurotrophic factors, such as vascular endothelial growth factor (VEGF) and glial cell-derived neurotrophic factor (GDNF), and provide neuro-restorative effects to the host microenvironment.

Despite the promising results from stem cell therapy, there remains a lack of understanding on the effector mechanism of the transplanted cells on endogenous neural progenitors. Particularly, the influences of cell-cell interactions on the trophic and secretory functions of exogenous and endogenous populations are poorly understood.

Recently, it was realized that implanted stem cells exert their clinical benefit largely via their secretome. In particular, extracellular vesicles (EVs) secreted from hMSCs or NPCs derived from hiPSCs have been shown to attenuate ischemic stroke and other diseases in both small and large animals. The injected EVs are thought to directly signal to the endogenous neural progenitors and the supporting cells, including endothelial cells, pericytes, and microglia, altering their response to ischemic injury. However, published studies have only used EVs from undifferentiated or partially differentiated cells, most of which are cultured as monolayers and lack the distinct miRNAs important in neurogenesis and 3-D brain tissue development.

### BRIEF SUMMARY

There is a need to understand the composition and function of EVs derived from brain organoids in order to improve post-ischemic stroke treatment.

There also remains a need for improved methods for generating EVs, including methods that have increased yields, have a cargo profile with higher therapeutic potential, are scalable, or a combination thereof.

There also remains a need for methods for fabricating next-generation EVs from human stem cells for drug delivery, cell-free therapeutics, or a combination thereof.

An example of a method includes producing extracellular vesicles (EVs) from human induced pluripotent stem cells (hiPSCs) by forming 3-D brain organoids of the hiPSCs in a dynamic bioreactor and collecting the EVs from the bioreactor.

This method may include one or more of the following features.

The dynamic bioreactor may be at least one of a wave motion bioreactor, a suspension bioreactor, and a vertical wheel bioreactor.

The method may further include encapsulating the collected EVs in a cross-linked hydrogel.

The cross-linked hydrogel may be a heparin-hyaluronic acid hydrogel.

The brain organoids may be forebrain cortical organoids and/or hindbrain cerebellar organoids.

Forming 3-D brain organoids of the hiPSCs in a dynamic bioreactor may include providing neural spheroids in a hydrogel and inoculating the neural spheroids in the presence of cyclopamine and FGF-2.

The EVs may include miRNA that contributes to neurogenesis.

The EVs may promote recovery after an ischemic injury.

An example of a product includes a pharmaceutical dosage form comprising extracellular vesicles (EVs) of brain organoids from human induced pluripotent stem cells (hiPSCs).

This product may include one or more of the following features.

The pharmaceutical dosage form may include the EVs in a cross-linked hydrogel.

The cross-linked hydrogel may be a heparin-hyaluronic acid hydrogel.

The brain organoids may be forebrain cortical organoids and/or hindbrain cerebellar organoids

The EVs may include miRNA that contributes to neurogenesis.

The EVs may promote recovery after an ischemic injury.

The pharmaceutical dosage form may be configured to inject the EVs into a patient.

An example of a method of treatment includes administering to a patient in need thereof a pharmaceutical dosage form comprising extracellular vesicles (EVs) of brain organoids from human induced pluripotent stem cells (hiPSCs).

This product may include one or more of the following features.

The patient may have an ischemic condition.

The pharmaceutical dosage form may include the EVs in a cross-linked hydrogel.

The cross-linked hydrogel may be a heparin-hyaluronic acid hydrogel.

The brain organoids are forebrain cortical organoids and/or hindbrain cerebellar organoids.

The EVs may include miRNA that contributes to neurogenesis.

The EVs may promote recovery after an ischemic injury.

Administering the pharmaceutical dosage form may include injecting the pharmaceutical dosage form into the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph characterizing neural spheroids derived from human iPSK3 cells by K+ and Na+ currents (day 50 Purmo-cells).

FIG. 1B is a graph characterizing neural spheroids derived from human iPSK3 cells by spontaneous post-synaptic currents (day 30 Purmo-cells).

FIG. 1C is a graph characterizing neural spheroids derived from human iPSK3 cells by cyclo-treated cells and Purmo-treated cell phenotype (day 35). Purmo activates SHH signaling which is required for hindbrain patterning. Cyclo inhibits SHH and is required for forebrain patterning.

FIG. 1D is a set of graphs characterizing neural spheroids derived from human iPSK3 cells by RT-PCR analysis (day 35), *p<0.05.

FIG. 2A is a set of phase contrast images of cortical organoids derived from a spinner bioreactor or in a 24 well plate (static).

FIG. 2B is a set of confocal images of aggregates with cortical layer markers (TBR1 and SATB2 at day 71) where the dashed lines show layer separation.

FIG. 2C is a graph of RT-PCR analysis of HOXB4, HB9, TBR1, vGluT1 (day 32).

FIG. 2D is a graph of flow cytometry analysis of SATB expression (day 36).

FIG. 2E is a graph of RT-PCR analysis of MMP2 and MMP3 (day 32), *p<0.05.

FIG. 3A is a graph characterizing hiPSC-EVs by average exosome size.

FIG. 3B is data characterizing hiPSC-EVs by (i) particle size distribution for iPSC-NP group measured by NTA and (ii) a representative electron microscopy image.

FIG. 3C shows Western blot data for exosomal markers. WCE: whole cell extracts. HEK293 cells as positive control. * , ** , and # indicate p<0.05 between the compared conditions and the undifferentiated iPSK3 group (n=3).

FIG. 4A is data showing therapeutic effects of hiPSC-EVs and hMSC-EVs by the effects of hiPSC-EVs on reducing oxidative stress of iNPC spheroids (day 30) treated with Aβ42 oligomers and hiPSC-EVs. The graph shows quantification of ROS expression (n=3), *p<0.05 compared to control.

FIG. 4B is data showing therapeutic effects of hiPSC-EVs and hMSC-EVs by the effects of hiPSC-EVs on reducing oxidative stress of iNPC spheroids (day 30) treated with Aβ42 oligomers and hiPSC-EVs. The graph shows flow cytometry analysis for ROS expression. The red line indicates iNPCs treated with Aβ42 oligomers (positive control). The blue line indicates iNPCs treated with Aβ42 oligomers plus hiPSC-EVs. MFI: mean fluorescence intensity

FIG. 4C is data showing therapeutic effects of hiPSC-EVs and hMSC-EVs by the effects of hiPSC-EVs on reducing oxidative stress of iNPC spheroids (day 30) treated with aβ42 oligomers and hiPSC-EVs. The graph shows miRNA cargo for 2D-EVs and 3D-EVs from hMSCs.

FIG. 4D is data showing therapeutic effects of hiPSC-EVs and hMSC-EVs by the effects of hiPSC-EVs on reducing oxidative stress of iNPC spheroids (day 30) treated with Aβ42 oligomers and hiPSC-EVs. The graph shows the anti-inflammatory effects of macrophage (M1 or M2) of hMSC-EVs. LPS: lipopolysaccharide.

FIG. 5 is a table indicating the representative markers for each cerebellar layer.

FIG. 6 is a graph of the RT-PCR analysis of miR-133 and miR-155 in different iPSC-EVs. Normalized to SNORD44. *p<0.05.

FIG. 7A is an illustration of the influence of Wnt signaling on neural tissue identity.

FIG. 7B is a Western blot of ubiquitinated β-catenin and β-catenin in mouse ES-D3 cells grown on different ECMs (AGG, EB, and NPC).

FIG. 7C is data showing the expression of HOXB4 (hindbrain marker) and TBR1 (forebrain cortical marker) in 3-D neural tissues derived from human iPSK3 cells regulated by CHIR99021 (CHIR). (i) is a set of TBR1 and HOX4 images. (ii) is a graph of RT-PCR analysis of TBR1 and HOXB4 (day 23), *p<0.05.

FIG. 8A is data showing lineage-specific ECM secretion by 2-D electrophoresis of endogenous ECMs from different groups. Green circle: proteins in AGG labeled with Cy2. Yellow circle: proteins in both AGG and NPC.

FIG. 8B is data showing lineage-specific ECM secretion by a Venn diagram representing the number of protein identifications found in each ECM group.

FIG. 8C is a table listing ECM proteins for specific NPC.

FIG. 9 is a schematic illustration of crosstalk of miRNAs and Wnt signaling.

FIG. 10A is data showing in vitro MRI analysis of MPIO-labeled NPCs as an illustration of the cell-agarose layers in a tube for in vitro MRI analysis.

FIG. 10B is data showing in vitro MRI analysis of MPIO-labeled NPCs as a 3-D gradient recalled echo (GRE) images if immobilized NPCs at day 1, 3, and 5.

FIG. 10C is data showing in vitro MRI analysis of MPIO-labeled NPCs as a relaxation graph showing T2.

FIG. 10D is data showing in vitro MRI analysis of MPIO-labeled NPCs as a relative signal for each MPIO exposure over 5 days, *p<0.05.

FIG. 11 is a graph of Hep-HA hydrogels with different modulus prepared by photo-crosslinking of methylated HA and thiolated heparin.

FIG. 12 is a high resolution ²³Na Chemical Shift Imaging (CSI) (right) processed in Matlab with an ischemic lesion in red compared to traditional ¹H T2-weighted image (left) 1 day post-surgery with immediate hMSC administration.

### DETAILED DESCRIPTION

Described herein are EVs of brain organoids from human iPSCs (iNPCo) methods of generating them, and methods using them. Certain examples of hiPSC-derived forebrain organoids may be used to generate tissue-specific EVs due to their unique ability to coordinate signaling that may drive the formation of tissue-like structures reminiscent of organization in vivo.

Also discussed herein are investigations into the impact of iNPCo-derived EVs on cell survival, extracellular matrix (ECM) and tropic factor secretion, and functional neural differentiation in vitro and in vivo. iNPCo, unlike naïve iPSCs and monolayer NPCs, may secrete EVs carrying brain-specific miRNA cargo that can target ischemic injured brain tissue both by providing protection from injury and/or by promoting recovery after injury; in particular, heparin-hyaluronic acid (Hep-HA) hydrogel encapsulation may allow for the sustained delivery of iNPCo-EVs in an ischemic environment, thereby promoting their therapeutic effects.

Human stem cells may be assembled into organoids in various types of bioreactors. A novel organoid culture method in dynamic bioreactor systems may be used to promote the secretion of EVs. The three dimensional cultures include, but are not limited to, forebrain organoids from hiPSCs, cerebellar organoids form hiPSCs, choroid Plexus organoids, and hMSC spheroids.

Not wishing to be bound by any particular theory, it is believed that, compared to previously used NPC-EVs, iNPCo-EVs may offer one or more of the following advantages: 1) the formation of 3-D organoids may up-regulate 20) the secretion of trophic, anti-apoptotic, and anti-inflammatory factors; 2) cell-free implantation may eliminate concerns of tumorigenicity from undifferentiated hiPSC fractions; 3) similar to other cell types including hMSCs, brain organoids (containing glial cells, microglia, etc.) may have an active secretome and can generate abundant EVs capable of regulating the phenotype of target cells, including microglia, astrocytes, pericytes etc.; and/or 4) iNPCo-EVs may be packaged with miRNAs that have specific influence on brain tissue development and repair.

Not wishing to be bound by any particular theory, it is believed that iNPCo, unlike naïve hiPSCs and monolayer NPCs, may secrete EVs carrying brain-specific miRNA cargo that can target ischemic injured brain tissue both by providing protection from injury and by promoting recovery after injury. Hydrogel encapsulation, such as heparin-hyaluronic acid (Hep-HA) hydrogel encapsulation, may allow for the sustained delivery of iNPCo-EVs in the ischemic environment, which may promote their therapeutic effects.

Not wishing to be bound by any particular theory, it is believed that EVs derived from hiPSC-forebrain organoids may better recapitulate the paracrine signaling of the human brain. In particular, forebrain organoids derived from hiPSCs may allow modulation of the EV microenvironment to recreate in vivo like conditions that drive normal development and regeneration.

An example of a composition includes a pharmaceutical dosage form including extracellular vesicles (EVs) of brain organoids from human induced pluripotent stem cells (hiPSCs).

The term “organoid” as used herein refers to a three dimensional mass of stem cells that can perform certain functions of an organ. Organoids may include 3-D structures such as spheroids and the like.

In the composition, the brain organoids may be forebrain cortical organoids and/or hindbrain cerebellar organoids, for example.

The EVs may include miRNA cargo that that contributes to neurogenesis. As such the EVs may be used to promote recovery after an ischemic injury.

The pharmaceutical dosage form may include the EVs in a cross-linked hydrogel. An example of such a cross-linked hydrogel is a heparin-hyaluronic acid hydrogel.

The composition may be administered to a patient in need thereof. Such a patient may have an ischemic condition such as after having experienced a stroke or other ischemic event.

The composition may be administered as an active ingredient in a pharmaceutical dosage form. In such a case, the composition may be blended with one or more ingredients useful for making the composition into a pharmaceutically acceptable dosage form such as a suspension, tablet, capsule, injectable, dermal patch, implant, or other dosage form.

Exemplary ingredients include one or more excipients, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

The composition may be therapeutically effective for treating an ischemic condition. A particular example of an ischemic condition is an ischemic stroke or other cause of cerebral ischemia.

The EVs may include miRNA that contribute to neurogenesis, 3-D brain tissue development, or a combination thereof.

The pharmaceutical dosage form may include a combination of different drugs and may include one or more additional active ingredients that are therapeutically effective for treating an ischemic condition.

The pharmaceutical dosage form may be administered as part of a dose regimen that includes varying changes in the dose during the treatment period.

There are many different ways that the pharmaceutical dosage form may be administered to a patient. These administration techniques include, but are not limited to administering as a pharmaceutically acceptable dosage forms such as suspensions, tablets, suppositories, capsules, injectables, transdermals, implants, or the like. Additional suitable administration techniques may include oral, sublingual, buccal, intravenous, subcutaneous, transcutaneous, intramuscular, intracutaneous, intrathecal, epidural, intraocular, intracranial, inhalation, intranasal, or the like. Any combination of administration techniques may also be used.

An oral dosage form such as a pill includes the composition combined with conventional excipients for tablet, capsule, or other pill-type dosage forms. The pill dosage form may be monolithic or particulate. Typical pill excipients may include binders such as sugars, gelatin, cellulose, starch, methyl cellulose, ethyl cellulose, hydroxypropyl methyl cellulose, and the like. They may also include fillers such as lactose, sucrose, cellulose, calcium carbonate, and the like. The pill may be formulated for extended or immediate release. If needed, the pill may be enteric coated.

An injectable dosage form may include the composition in a liquid carrier such as saline, oil, alcohol, or the like, optionally combined with a surfactant to aid solubility or emulsification of the composition. It is to be understood that injectable dosage forms include implant that can be placed on the body or inside the body and are capable of injecting the composition into the body.

In certain examples, the pharmaceutical dosage form is configured for intra-arterial injection or intra-cerebral injection.

The pharmaceutical dosage form may include a hydrogel in which the EVs are dispersed.

The pharmaceutical dosage form may be configured for injection or implantation, and, optionally, after injection or implantation, the pharmaceutical formulation may releases the EVs at a controlled rate, for example, a sustained rate.

Suitable administration techniques include any of the aforementioned administration techniques and associated pharmaceutical dosage forms.

The therapeutically effective amount can vary and be adjusted to the patient's needs. By way of example, a daily dosage of between about 0.01 and about 100 mg/kg body weight per day may be appropriate. A particular daily dosage range may be 0.1 to 500 mg/kg body weight, 0.1 to 250 mg/kg body weight, or 0.1 to 100 mg/kg body weight per day.

If the dosage form includes a solution containing the composition, the concentration of the composition may be, for example, about 0.01 μM to about 1,000 μM, about 1 μM to about 500 μM, or about 10 μM to about 250 μM.

An example of a method of making the composition includes producing EVs from hiPSCs by forming 3-D brain organoids of the hiPSCs in a dynamic bioreactor and collecting the EVs from the bioreactor.

A dynamic bioreactor is a dynamic culture system as opposed to a static culture system. Examples of dynamic bioreactors include, but are not limited to, a wave motion bioreactor, a suspension bioreactor, and a vertical wheel bioreactor. Using a dynamic bioreactor may result in increased EV yields and/or improved therapeutic cargo, such as miRNA, profiles.

In certain examples of this method, forming 3-D brain organoids of the hiPSCs in a dynamic bioreactor by providing neural spheroids embedded in a hydrogel and inoculating the neural spheroids in the presence of cyclopamine and FGF-2.

The EVs may be collected using conventional techniques.

In some examples, the EVs may be isolated by subjecting the culture to differential ultracentrifugation.

### EXAMPLES

The following examples are not to be construed in any way as imposing limitations upon the scope of the claims.

The work of Examples 1-3 establishes a platform for iNPCo-EV based stroke therapy. The examples determine the effects of paracrine signaling on neural regeneration and establish a transformative approach for stroke therapy by modulating the extracellular microenvironment to attenuate ischemia-associated neuropathology.

### Example 1-iNPCo-EVs Expression of Exosomal Markers and Promotion of Cell Survival Under Oxidative Stress In Vitro

In this example, forebrain cortical organoids (iNPCo) and hindbrain cerebellar organoids were derived from iPSCs. Then, characterizations were performed for cortical layer and cerebellar structure (by confocal microscopy, histology, transcriptome analysis, etc.) and neural function by electrophysiology. EVs from the culture medium or decellularized matrices were isolated and characterized for size distribution (nanoparticle tracking analysis), electron microscopy for morphology, and exosomal markers CD63,TSG101, Hsc70, and Alix (Western blot).

The exosome (pre-labeled) uptake was examined using isogenic cortical spheroids. Cell viability, reactive oxygen species, caspases, and anti-inflammatory ability were examined. iNPCo-EVs should exhibit better neural protection ability and reduction of oxidative stress.

EVs from isogenic 2-D NPCs, MSCs, and undifferentiated iPSCs were used as controls.

### Background and Rationale

(a) 3-D Forebrain Organoid Models Derived from hiPSCs

Brain tissues were modeled following the formation of serum-free, floating embryoid body (EB)-like aggregates from hPSCs. In the presence of fibroblast growth factor FGF-2 and inhibitors of bone morphogenetic proteins, Wnt/I3-catenin, and transforming growth factor (TGF)-I3/activin/nodal pathways, hiPSCs generate 3-D structures that contain polarized glia intermediate progenitors and layer-specific cortical neurons. More recently, cerebral organoids have been formed using hiPSC-derived NPCs which show regional specification of brain structures, functional neural activity, and the presence of glia cells. Focusing on stroke treatment, 3-D forebrain organoids of this example are compared with 2-D NPCs and hindbrain cerebellum organoids for EV generation.

(b) Effects of hiPSC-EVs:

hiPSC-EVs, enriched with proteins and miRNAs, are safer (no tumor formation) than parent hiPSCs for tissue repair in vivo. hiPSC-EVs restored cell viability and capillary-like structure formation, and reduced senescence in human endothelial cells exposed to high glucose. hiPSC-EVs were also reported to stimulate the proliferation and migration of human fibroblasts.

In addition, iPSC-EVs reduced MMP-1/-3 and restored the expression of collagen I in senescent fibroblast cells. In particular, hiPSCEVs can reduce reactive oxygen species (ROS) levels of senescent hMSCs, improve the growth of replicatively aged hMSCs and alleviate cellular aging in part by delivering peroxiredoxin antioxidant enzymes (e.g., PRDX1/2). ALIX overexpression (using Crispr/cas9 genome editing) was reported to increase therapeutic function of iPSC-secreted exosomes, indicated by protective effects on injured endothelial cells and rescuing hydrogen peroxide (H2O2)-blocked angiogenesis. hiPSC-NSC EVs have neuroprotective, anti-apoptotic, antioxidant, anti-inflammatory, neurogenic, and blood brain barrier requiring activities.

### Data:

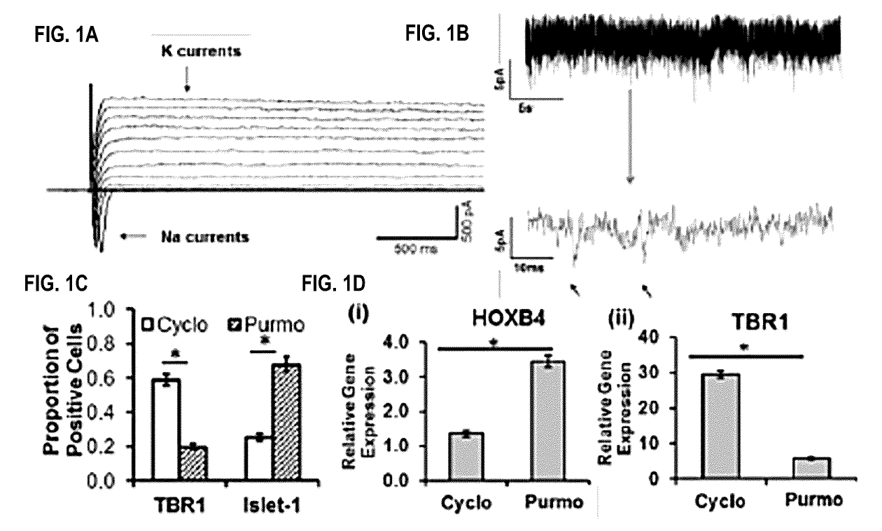

(a) Forebrain organoids derived from hiPSCs: Brain spheroids of human iPSK3 cells were generated in DMEM/F-12 plus B27 medium supplemented with dual-SMAD inhibitors LDN193189 (0.1 μM) and SB431542 (10 μM) (FIG. 1). Neural tissue patterning was tuned through treatment with the antagonist of sonic hedgehog (SHH) signaling cyclopamine or agonist purmorphamine, along with other factors, such as FGF-2, retinoic acid (RA), and further maturation. Quantitative RT-PCR analysis confirmed the neural patterning effects of cyclopamine (e.g., higher TBR1) and purmorphamine (e.g., higher HOXB4) (FIG. 1C, 1D). Characterization of electrophysiology with whole-cell patch-clamp recordings showed an inward voltage-gated Na+current followed by a more sustained K+current (day 50) (FIG. 1A) and spontaneous excitatory post-synaptic currents (as early as day 30) (FIG. 1B). These data demonstrate the feasibility of deriving functional cortical organoids from hiPSCs for EV isolation.

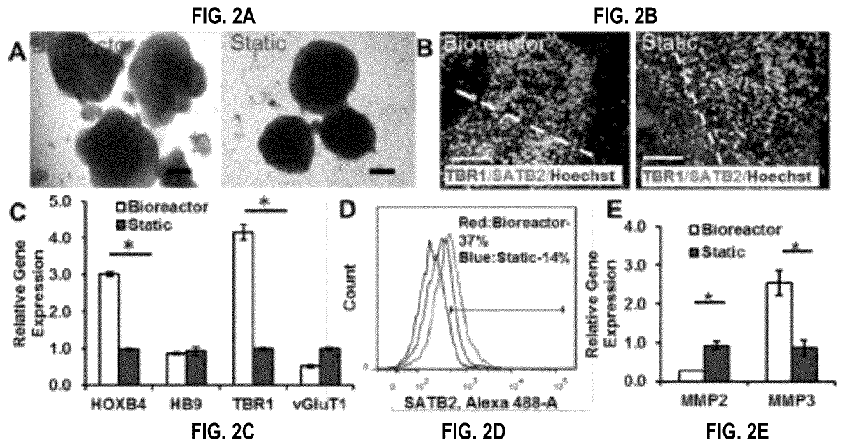

Maturation of cortical spheroids (more than 1 mm in diameter) derived from hiPSK3 cells were performed in a spinner bioreactor compared to a static 24-well plate culture (FIG. 2A). The deep cortical layer of TBR1+ cells (layer VI) appeared first, followed by the SATB2+ (layers II-IV) superficial layer cells (according to “inside-out” cortical developmental pattern), showing the identity of cortex tissue (FIG. 2B). Prolonged culture to day 71 showed distinct cortical layer specific structure (FIG. 2B). The spheroids in bioreactors demonstrated faster cortical tissue development indicated by the higher TBR1 (by RTPCR analysis) and SATB2 (by flow cytometry analysis) expression compared to the static culture (FIG. 2D, 2E). These data demonstrate the feasibility of maturing NPC spheroids into cortical organoids generated from hiPSCs.

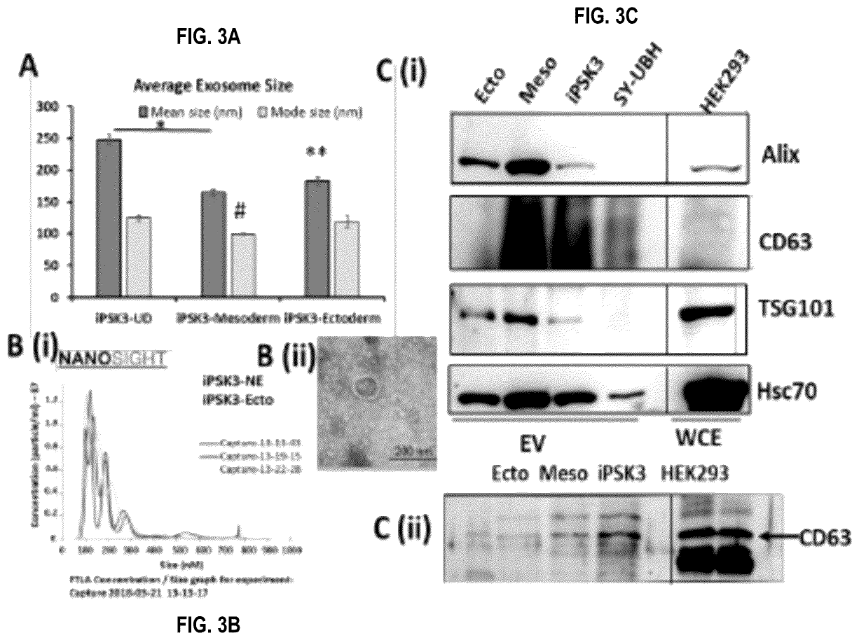

(b) Characterizations of iNPCo-EVs: Nanoparticle tracking analysis (NTA) was performed for EVs isolated from spent media of undifferentiated iPSK3 cells, iPSK3-mesoderm cells (day 15-20), and iPSK3-neural progenitors (NP: day 15-20) using a Nanosight LM10-HS. The EVs were isolated using the Total Exosome Isolation Kit and the ExtraPEG method. The average size of NP ectoderm-EVs was 182.6±7.2 nm (mode size 119.1±9.4 nm) (FIG. 3A, 3B(i)).

Small round particles with cup-shaped morphology were identified in all samples, verifying that exosomes were harvested (FIG. 3B(ii)). Alix and TSG101 are part of the endosomal sorting complexes required for transport (ESCRT) machinery and CD63 is a tetraspanin common on the surface of exosomes. Hsc70 is a heat shock protein. These exosomal markers were detected in iPSK3 derived-EVs (FIG. 3C). SY-UBH line was derived from the fibroblasts of an early-onset Alzheimer's individual with Presenilin 1 M146V mutations. The secreted EVs had low levels of Alix and TSG101, probably due to neural degeneration. These results demonstrate the feasibility of isolating and characterizing EVs from hiPSCs at different lineage specifications and genetic backgrounds.

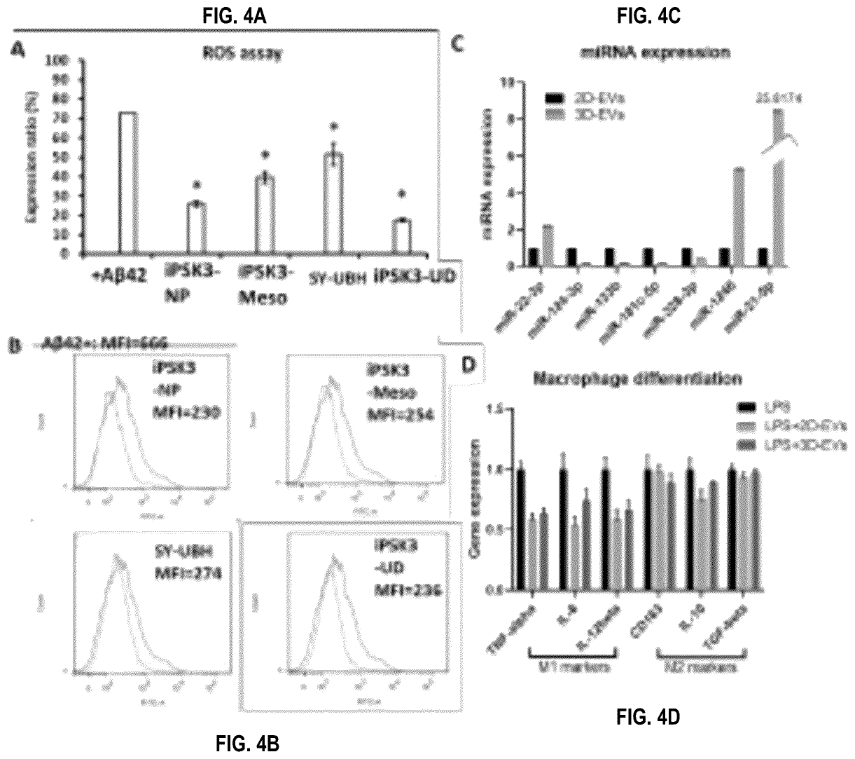

(c) Biological effects of iNPC-EVs in vitro: Undifferentiated iPS K3-EVs, undifferentiated SYUBH-EVs, iPSK3-mesoderm EVs, and iPSK3-ectoderm EVs were added to day 16 iPSK3-derived cortical spheroids treated with 1 μM Aβ42 oligomers. Compared to no EV control, treatment of EVs from undifferentiated iPSK3, iPSK3-mesoderm, and iPSK3-ectoderm reduced the expression of ROS (FIG. 4).

However, the treatment of SY-UBH derived EVs still retained high ROS expression. These results demonstrate the antioxidant effects of EVs secreted by hiPSCs at different lineage specifications. For hMSCs, the 3D-EVs contained higher levels of miR-21-5p and miR-1246, and

(d) RNA-seq for dorsal cortical organoids: Transcriptome analysis was performed on the dorsal cortical organoids containing isogenic microglia like cells, in comparison to microglia-like cells. The genes of astrocytes, oligodendrocytes, pericytes, and microglia were observed, suggesting the presence of multiple cell types in these forebrain organoids. These results indicate that the secreted EVs may better represent homeostatic brain tissue.

### Methods:

1. Undifferentiated hiPSCs (group I): Human iPSK3 cells were maintained in mTeSR serum-free medium on Matrigel-coated culture vessels. The cells were passaged by Accutase every 4-6 days in the presence of Y27632 (10 μM). A human iPSK3 cell line was derived from human foreskin fibroblasts transfected with plasmid DNA encoding reprogramming factors OCT4, NANOG, SOX2 and LIN28. The conditioned media from day 4-7 cultures were collected 24 hrs after medium change. Additionally, two healthy hiPSCs lines (commercial ATCC-HYR0103 and ThermoFisher A18945) were included in this study.

2. HiPSC differentiation into NPCs in 2-D culture (group II): Human iPSK3 cells were seeded into tissue culture treated 24-well plates at 3×105 cells/well in 1 mL of differentiation medium composed of DMEM/F-12 plus 2% B27 serum-free supplement. ROCK inhibitor Y27632 (10 μM) were added during the seeding and removed after 24 hours. At day 1, the cells were treated with dual SMAD inhibitors SB431542 (10 μM) and LDN193189 (100 nM). After 8 days, the cells were treated with FGF-2 (10 ng/ml) and a sonic hedgehog antagonist cyclopamine (1 μM) until day 16. The conditioned media from day 15-20 cultures were collected.

3. HiPSC differentiation into forebrain cortical organoids (group III): Briefly, hiPSCs were seeded into low attachment plates in DMEM-F12 plus 2% B27 medium in the presence of LDN193189 (100 nM) and SB431542 (10 μM). At day 8, the neural spheres were resuspended in Matrigel (0.1-1 mg/mL, as control) or in-house made ECM hydrogels (e.g., Hep-HA hydrogels, VitroGel) (0.1-1 mg/mL). After the gels were formed at 37° C., the spheres were grown in DMEM/F-12 and B27 in the presence of cyclopamine (1 μM) and FGF-2 (10 ng/ml). This protocol is designed to obtain dorsal telencephalic progenitors, which predominately differentiate into cortical glutamatergic neurons in forebrain organoids. Neural spheroids (day 15) embedded in hydrogels were inoculated into spinner bioreactors (Micro-Stir® Unit and CelStir® spinner flask: 25-50 mL) in media with cyclopamine and FGF-2. The bioreactor culture continued up to day 30-70 to mature cortical organoids. The conditioned media from day 15-20, 30-40, and 50-60 were collected.

4. HiPSC differentiation into hindbrain cerebellar organoids (group IV): Y27632 (10 μM) and TGF-131 inhibitor SB431542 (10 μM) were added to the culture. On day 2, FGF-2 (50 ng/ml) were added until day 7. During day 8-14, retinoic acid (1.0 μM) and a Wnt activatorCHIR99021 (10 μM) were added as well as ECM hydrogels. During day 15-21, FGF19 (100 ng/ml) were used to promote caudalization of neural rosettes. Sequentially, SDF-1A was added during day 28-35 to induce the molecular layer, the Purkinje cell layer, and the granule cell layer. Purmorphamine (2 μM) was also added to promote ventralization. The spheroids were transferred into spinner bioreactors and cultured up to day 70. The conditioned media from day 15-20, 30-40, and 50-60 were collected. In addition, hMSC-EVs (group V) were collected for the comparison in animal study.

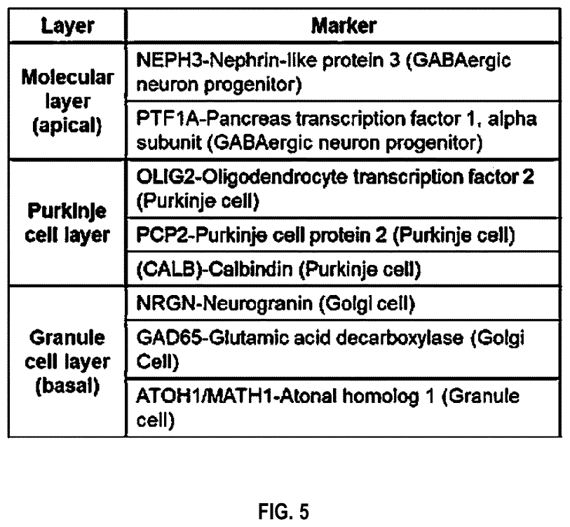

5. Brain organoid characterizations: Neural markers were evaluated using immunostaining, flow cytometry, Western blot, and RT-PCR: (1) general neuronal markers-I3-tubulin III, MAP2, and Neun; (2) cortical layer markers-TBR1 (deep layer VI), CTIP2 (layer V), SATB2 and BRN2 (superficial layer II-IV), glutamate, vGluT1, and GABA; (3) hippocampal markers-PROX1 and TBR1; (4) cerebellar organoids were characterized according to FIG. 5.

Histology of organoids were evaluated for cortical layer structure and regional specification of ventricular zone (VZ) and sub-VZ as well as for cerebellar layers. RNA-sequencing was performed for transcriptome analysis. To assess synaptic connectivity, synapsin I (a pre-synaptic marker) and PSD95 (a post-synaptic marker) are evaluated by immunocytochemistry. Moreover, the organoids were sliced into 250 μm for: (1) measuring spontaneous neuronal activities by Ca²⁺ imaging in cells loaded with Ca2+ indicator Fluo-4 AM or Cal-520AM [94]; (2) evaluating neuronal maturity by measuring Na+, K+ currents and action potentials using patch clamp recordings; and (3) determining synaptogenesis by recording spontaneous and electrical stimuli evoked synaptic activity. The gain and-loss assays were performed using tetrodotoxin to create action potential blockade. Electrophysiology (see FIG. 1) was performed.

6. Total EV isolation: Differential ultracentrifugation was used for EV isolation. An inexpensive polyethylene glycol-based method (ExtraPEG) was used along with Optipep density gradient to obtain highly purified small exosome preparations. Prior to any analysis, EVs were validated and characterized using standard endpoints as defined by the International Society of Extracellular Vesicles. These include vesicle characterization by at least two different methods (i.e., NTA and transmission electron microscopy) and the presence or absence of EV-specific or EV-lacking proteins by Western blotting. Purity was measured as described by Webber and Clayton.

7. EV characterizations for exosomal markers and morphology: Nanoparticle tracking analysis was performed on fresh EV samples in triplicate using a Nanosight LM10-HS (Malvern Instruments) configured with a blue (488 nm) laser and sCMOS camera to estimate size distribution and particle concentration. Samples were diluted to 1-2 μg/mL in particle-free software was used to measure the mode and mean size, as well as the concentration of particles per 1 mL solution. Western blot was performed for positive exosomal markers-CD9, CD63, CD81, TSG101, Hsc70, and Alix; as well as for negative exosomal marker-calnexin. Flow cytometry was performed for CD9 and CD63 (using magnetic beads).

For miRNA preservation, RNA was extracted using the Total Exosome RNA and Protein Isolation Kit (Thermo Fisher). Concentrations of 1,500 pg/μL were loaded to an Agilent 6000 pico chip and total RNA was quantified using an Agilent 2100 Bioanalyzer. The selected miRNA expression was compared for different EV groups. The target miRNAs include miR-133, miR-219 etc.

8. Cell survival under oxidative stress and anti-inflammation ability: Oxidative stress has been found to impact cellular redox status and may affect neural lineage commitment. Day 20-30 forebrain spheroids were subjected to B27 removal or exposure to H202, in the presence of different types of iPSC-EVs (labeled with DiI dye at 50-100 μg protein/mL), to determine the impacts of nutrient depletion and oxidative stress on cell survival. Cell viability was determined by Live/Dead assay. Cell apoptosis was determined by detecting caspase-3, -7, and -8 (caspase kits). Cell proliferation was evaluated by 5-bromo-2′-deoxyuridine (BrdU) labeling. The production of ROS was analyzed using carboxy-H2DCFDA incubation with inducer tertbutyl hydroperoxide (ROS detection kit). The anti-inflammatory ability was assessed using macrophage culture (M1 or M2).

Expected outcome: EVs derived from brain organoids may better promote cell viability and reduce oxidative stress compared to EVs of 2-D NPCs and hMSCs. The analysis, criteria and expected outcomes are shown in Table 1.

TABLE 1
Analysis and success criteria for the characterization of different hiPSC-EVs.

| Properties | Indicators | Methods | Expected outcome |
|---|---|---|---|
| Total proteins | Protein/mL spent | Protein assay | 1-10 ug protein/mL spent |
| | medium | | medium |
| Size distribution | Average EV/exosome | NTA | Size range [symbol] 50-300 nm |
| | size | Electron microscopy | |
| In vitro uptake | Exosomes internalized | Fluorescence labeled | >70% of EVs/exosomes |
| assays | by recipient cells | exosomes | are taken by the recipient |
| | | Flow cytometry | cells |
| Exosomal | CD9, CD63, CD81, | Flow cytometry | >70% positive CD9 [symbol] |
| markers | Hsc70, TSG101, and | Western blot | CD63 [symbol] strong positive |
| | Alix [symbol] Calnexin | | bands [symbol] negative Calnexin |
| miRNAs | miRNA preservation | Agilent 2100 | miRNAs are preserved |
| | e.g. miR-133 [symbol] miR-219 | Bioanalyzer | |
| Neural protective | Cell viability, | LIVE/DEAD | Increased viability [symbol] |
| ability | proliferation, ROS | ROS assay | Reduced ROS [symbol] |
| | levels for cells treated | BrdU assay | Identify the best EV group |
| | with H₂O₂ or starvation | | |

[symbol]

indicates data missing or illegible when filed

### Alternative Strategies

The yields of EVs are 2-3 μg protein per mL of spent medium. While this is sufficient, the yield of EVs may be enhanced using hollow-fiber bioreactor culture and medium perfusion. The cellular uptake of EVs may be determined using DiI dye and image analysis.

Purity of the EVs: The current isolation procedure co-purifies proteins and RNA complexes. To obtain highly purified EVs, the ExtraPEG method is combined, in some experiments, with Optipep density gradient.

Matrix-bounded EVs: the matrix derived from the brain organoids binds EVs as shown in recent publications. Alternatively, EVs are derived from decellularized ECMs of the brain organoids.

Data variability: To reduce the variability of results, the iPSC culture density and culture duration were well controlled. Particle-free reagents were used throughout the experiments.

### Example 2—To Test the Hypothesis that the microRNAs in iNPCo-EVs Regulate the Wnt Pathway and the Secretion of Trophic Factors and ECMs to Stimulate Neurogenesis In Vitro

In this example, miRNAs were isolated and the profiling was examined using miRNA-Seq technology for different groups of EVs. The focus was identifying the miRNAs that target the Wnt signaling pathway (e.g., miR-155), which is one of the specific signaling pathways that can influence human brain tissue development. Then the miRNA cargo was modified by transfection to obtain miR overexpressing cells (e.g., miR-155). The impact on Wnt signaling was examined by looking at 13-catenin, LRP5/6, and Frizzed receptor expression. The EV protein cargo was characterized by liquid chromatography-tandem mass spectrometry-based proteomics. miR inhibition may be achieved through pharmacological molecules (e.g., in the case of miR-155, MRG-106 is used). These examples demonstrate the feasibility of modifying miRNA cargo to deliver targeted signaling biomolecules.

Background and Rationale: A unique property of EVs is their ability to deliver multiple miRNAs that can act synergistically on separate processes to produce therapeutic effects. Thus, the ability to tailor the miRNA profiles of iNPCo-EVs allows, in some tests, optimization of their therapeutic benefits. MicroRNA profiling for hPSC-derived cortical neurons and oligodendrocytes shows the importance of miRNAs in neural cell function.

In particular, miRNAs regulate a majority of Wnt signaling components, which are regulators of tissue stiffness and disease development. For example, miR-221 targets the transcriptional factors in the canonical Wnt pathway and miR-155 targets 13-catenin interacting proteins. Wnt signaling regulates the rostral-caudal brain tissue identity, with high Wnt signaling leading to caudal identity and low activity leading to rostral identity.

In this example miRNA cargo was engineered in order to modulate Wnt signaling. EV engineering was achieved using molecular cloning and lentivirus packaging to fuse ischemic myocardium-targeting peptide with exosomal protein. Over expression of miR-146 also was achieved through transfection using hsa-miR-146b expression plasmids. Another possible method is to use anti-miR molecules.

### Data:

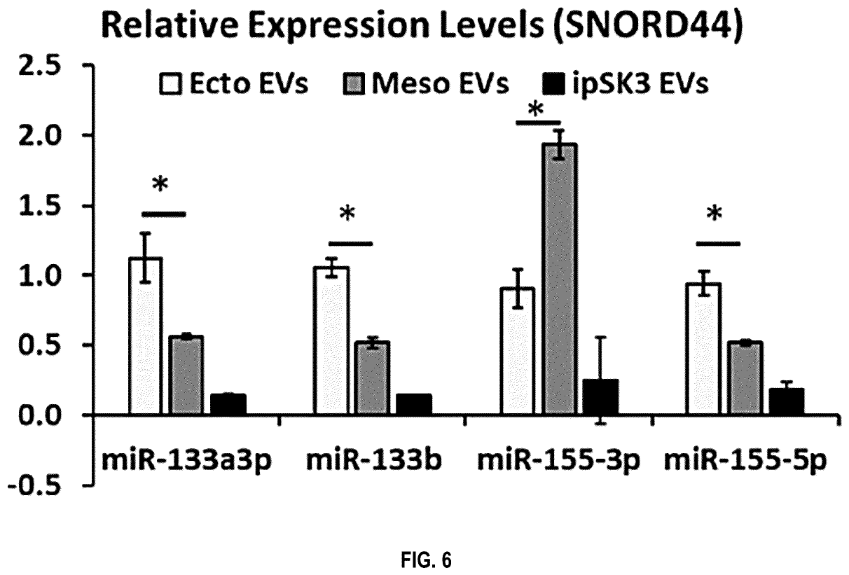

(a) miRNA isolation and detection from iPSC-EVs: Total miRNAs were isolated from different iPSC-EVs using the miRNeasy Micro Kit (Qiagen) according to the manufacturer's protocol. cDNAs were made using qScript miR CDNA synthesis kit (Quanta Bio). qPCR was performed for miR-133a, miR-133b, and miR-155 in different EV groups, showing differential expression (FIG. 6).

A similar study for hMSC-EVs was also performed. These data demonstrate the feasibility of characterizing miRNAs in iPSC-EVs to investigate the relationship of miRNAs with Wnt signaling.

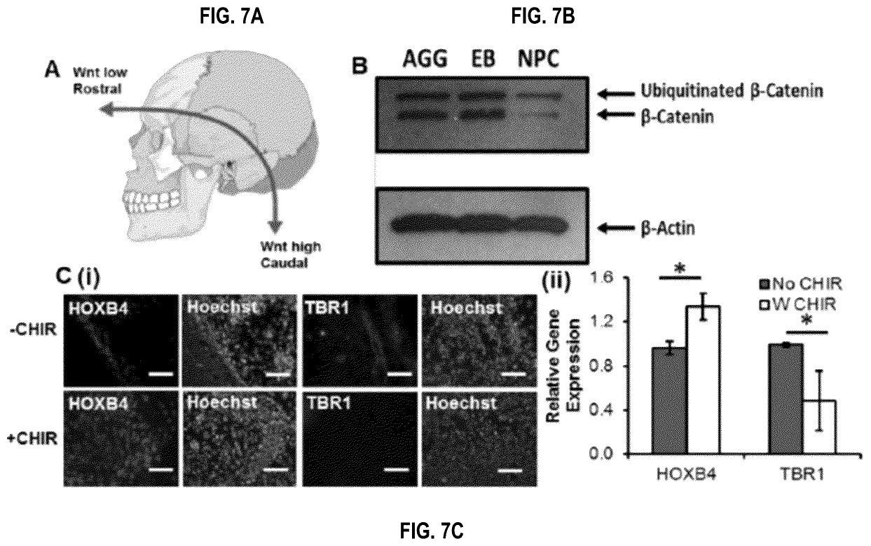

(b) Wnt/B-catenin signaling in neural differentiation: Wnt/β-catenin signaling plays a critical role in PSC lineage commitment, neural cell patterning, and neural tissue development. Therefore, the influence of Wnt singling on stem cell lineage commitment has been studied. Neural tissue patterning is influenced by Wnt signaling, which modulates rostral and caudal brain cell identity (FIG. 7A). To reveal the influence of ECMs on Wnt signaling, ES-D3 cells were seeded onto different types of ECMs derived from undifferentiated PSC aggregates (AGG), embryoid bodies (EB), and NPC aggregates (NPC). The Western blot data showed that ECMs derived from NPCs reduced the expression of β-catenin in the reseeded cells, indicating the down-regulation of canonical Wnt signaling (FIG. 7B).

These results demonstrate the influence of ECMs on Wnt signaling during early-stage ectoderm differentiation from PSCs. At this stage, Wnt inhibition enhances neurectodermal differentiation. During late-stage neural differentiation, the expression of HOXB4, the caudal marker for hindbrain/spinal cord, was upregulated in the presence of a Wnt activator CHIR99021, while in the absence of Wnt activation, the cells maintained rostral forebrain identity (expression of TBR1, a marker of deep cortical layer VI) (FIG. 7C). These data demonstrate the feasibility of evaluating the influence of Wnt/B-catenin signaling on human brain tissue patterning.

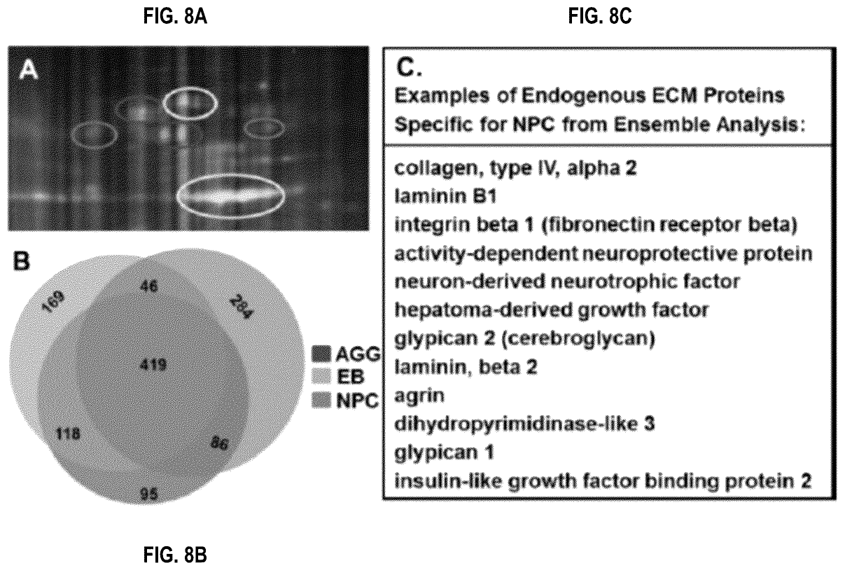

(c) Proteomics analysis: To further define the ECM secretion profile, liquid chromatography-mass spectrometry (LCMS)/MS and 2-D electrophoresis were used to characterize endogenous ECMs derived from undifferentiated PSC aggregates (AGG), spontaneously differentiated EBs (EB), and embryonic stem cell (ESC)-derived NPC aggregates(NPC). 2-D electrophoresis (FIG. 8A) showed differential expression of the proteins in AGG and NPC which were tagged with fluorescent dye Cy2 (green) and Cy5 (red) respectively. ECM samples were also analyzed using an MS-based proteomics protocol.

Numbers within the Venn diagrams represent the total number of protein identifications found in each ECM group and the specific overlap (FIG. 8B). ECMs from NPC samples displayed more distinct differences (27%) compared to the ECMs from EB or AGG groups (1216%). Data sets were also filtered using gene ontology classification based on the term “extracellular proteins.” Representative proteins identified in ECMs derived from NPC are shown in FIG. 8C. These data demonstrate the feasibility of evaluating ECM protein profiles using proteomics tools.

### Methods:

1. Proteomics analysis of EV protein cargo: Proteomics analysis on the different EV groups and the EV treated hiPSC-cortical organoids (2 cell lines at day 30 and 60) was performed. Proteins were extracted using a filter-aided sample preparation method with some modifications. The protein samples from 6 biological replicates were analyzed on an Orbitrap Q Exactive HF using a Thermo Easy-nLC-II separation. Following injection, the samples were loaded onto anEASY C18-A1 guard column for desalting; retained peptides were separated on a Thermo EASY C18-A2 column. Data analysis was performed using Scaffold proteome software and the Ensemble database.

Data sets were filtered using gene ontology classification for cellular components based on the term “neural regeneration.” This example identifies the list of candidate proteins that are involved in cortical tissue development and neural regeneration.

Proteins uniquely identified from cell-type specific EVs or differentially expressed were confirmed by Western blot analysis using antibodies specific to the proteins of interests. Confirmed targets were analyzed further in rapid and sensitive EV isolation and detection techniques that could be developed into diagnostics platforms. These techniques include, immunoaffinity capture with anti-CD63, -CD9 and -CD81 conjugated magnetic beads, ExtraPEG precipitation of EVs, detection using an ELISA-based assays for exosomal surface proteins, and further confirmation using flow cytometry and florescent nanoparticle tracking analyses.

This example confirms differentially expressed EV components that are specific to particular lineage and therefore represent good candidates to be tested using EVs from the serum of patients or in preclinical models.

2. miRNA profiling of EV miRNA cargo: This example identifies the differentially expressed miRNAs in different EV groups, in particular those affecting Wnt signaling. To do so, the tests purified the miRNA from exosome samples using the Qiagen miRNA kit and generate libraries using the NEBNext multiplex small RNA library kit following validation of the purity and quantity of miRNA obtained. To ensure that the miRNAs were of vesicle origin, exosome samples were treated with RNase prior to purification. RNA samples were submitted for miRNA Deep Sequencing. Statistically significant differences between sample groups were validated by real-time PCR. miRNA cargo analysis was collaborated with Co-I Tristan Driscoll.

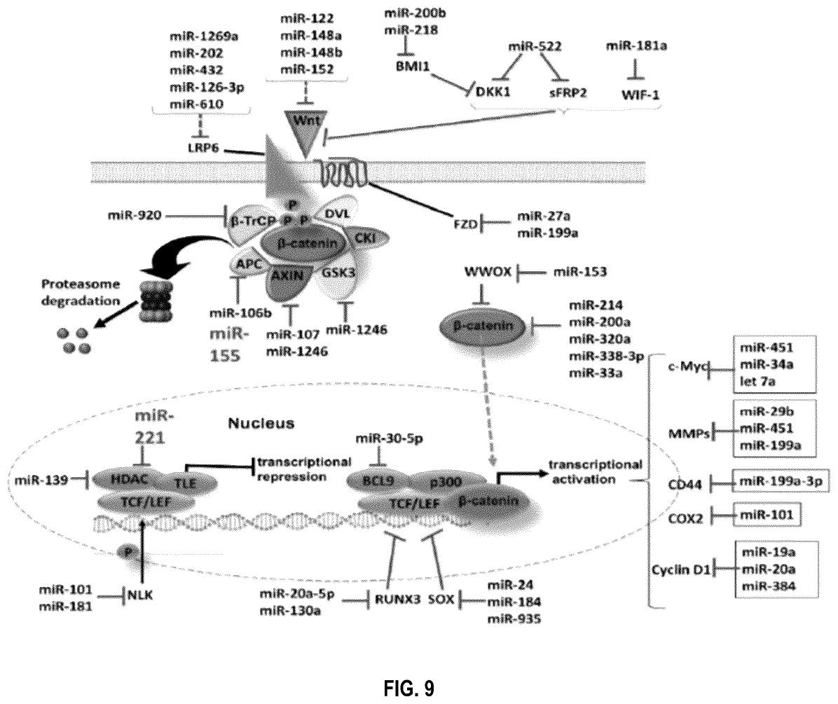

3. Influence of miRNA cargo on canonical Wnt signaling: The overexpression of exosomal miRNAs that affect Wnt signaling in the producing cells generated EVs that promote neural patterning of receiving cells (FIG. 9).

Through hiPSC genetic engineering, the corresponding EV cargo was modified. Specifically, cel-miR-67, hsa-miR-155, and hsa-miR-146b expression plasmids (GenScript) were used. iPSK3 cell transfection was performed using electroporation. About 2×106 cells were suspended in 150 ∥ of Ingenio Electroporation Solution (Mirus) with 2 μg of plasmid DNA. Program A-33 was used for electroporation in an Amaxa Nucleofector Device.

Transfected cells were resuspended in 10 mL complete culture medium, centrifuged, and then plated for exosome production. The genetically engineered cells were characterized for the expression of B-catenin, LRP5/6, and Frizzed receptor (Western blot or RT-PCR). The secreted EVs were isolated and used to treat normal organoids (derived from non-engineered iPSCs) to test if cortical/cerebellar layer separation and patterning were promoted (i.e., rostral-caudal axis and cell identity).

4. Engineering miRNA cargo by modulation of Wnt signaling: The Wnt signaling pathway has been proposed as a potential target for treating neurological diseases, as in vivo activation of Wnt signaling reverses the cognitive deficits. This example tests whether miRNA cargos are affected by modulation of Wnt signaling in the 3-D brain organoids. Wnt signaling is altered using GSK-313 inhibitors CHIR99021 and SB415286, 13-catenin mediated transcription inhibitor XAV-939, or Wnt inhibitor IWP4 or DKK-1 at different doses (0.01-5 μM).

The secreted EVs were isolated and the influence of Wnt signaling on miRNA cargo was evaluated through RT-PCR or miRNA profiling. Wnt activation using small molecules reduced miRNAs that inhibit Wnt signaling. The EVs were added to the cells treated with H2O2 or B27 starvation. The readouts included cell viability, ROS generation (by fluorescent assay), caspase expression, synaptic density, and functional synaptic activity (by electrophysiology).

5. miRNA inhibition: miR-155 inhibition was achieved by a locked nucleic acid MRG-106 (miRagen Inc.). MRG-106 treatment inhibits canonical Wnt signaling. Therefore, the organoid cultures treated with MRG-106 are characterized for Wnt-associated molecules and the downstream targeted genes. Another approach for miRNA inhibition is to use anti-miRs, such as anti-miR-221.

6. Secretion of trophic factors and ECMs for cortical organoids treated by engineered EVs: To evaluate trophic function, conditioned media was collected from cortical organoids treated with different groups of EVs at day 1, 3 and 5, and analyzed for FGF-2, BDNF, TGF-131, VEGF, PDGF-B, and IGF-1 by ELISA and Western blot.

The results were normalized to cell numbers. For the endogenous ECM secretion of EV-treated cortical organoids, ECM proteins including fibronectin, laminin, HA, chondroitin sulfate proteoglycans, collagen IV, and vitronectin are characterized by immunostaining with confocal microscopy and quantified by in situ ELISA. LS-MS/MS analysis was also used for analyzing endogenous ECM profiles of EV-treated cortical organoids.

Expected outcome: The cells should secrete different profiles of miRNAs in response to the organoid structure. The interactions of miRNAs with Wnt/β-catenin signaling plays an important role in human brain tissue development. The engineered miRNA cargo in EVs can promote neuronal function in vitro.

The analysis, criteria and expected outcomes are shown in Table 2.

TABLE 2
Analysis and success criteria for EV-miRNA targets of Wnt signaling.

| Properties | Indicators | Methods | Expected outcome |
|---|---|---|---|
| miRNA cargo | Differential | miRNA-sequencing | Differentially expressed |
| | expression of | miRNA profiling | miRNAs that affect Wnt |
| | different miRNAs | | are identified in different |
| | | | EV groups. |
| Wnt activation by | β-catenin, | Western blot [symbol] RT-PCR [symbol] | β-catenin, LRP5/6, and |
| miR-155 | LRP5/6 [symbol] and | Histology, flow cytometry | Frizzed, are upregulated: |
| overexpression | Frizzed [symbol] | for (TBR1, SATB2, | More caudal identity in |
| | Rostral-caudal | FOXG1, BRN2, Olig2, | the spheroids |
| | identity | OTX2) | |
| miRNA | miRNA content | Wnt activation by | Wnt activation reduces |
| modulated by | (composition) | CHIR99021 [symbol] SB415286 [symbol] or | Wnt-targeting miRNAs [symbol] |
| Wnt | | XAV-939, or Wnt inhibition | Wnt activation reduces - |
| | | by IWP4 or DKK-1 [symbol] | H₂O₂-induced |
| | | With H₂O₂ or starvation | neurotoxicity |
| miRNA inhibition | Wnt-associated | Use MRG-106 to inhibit | Wnt-associated |
| | molecules and | miR-155 [symbol] | molecules are |
| | the downstream | Western blot | downregulated |
| | targeted genes | RT-PCR [symbol] genomics | |
| miRNA blocking | Wnt-associated | Use anti-miR-221 | Wnt-associated |
| | molecules | to inhibit | molecules are |
| | | miR-221 | downregulated |

[symbol]

indicates data missing or illegible when filed

### Alternative Strategies

Quality control of organoids and the derived EVs is important to reduce data variability. The starting cell numbers, the raw reagents, and EV isolation procedure may be controlled.

For the selection of miRNAs that target Wnt signaling, miR-155 and miR-221 were used as the examples of miRNAs that target Wnt signaling based on literature. The candidate miRs could be revised based on our miRNA profiling results.

Wnt signaling can crosstalk with other signaling pathways such as Notch and Hippo/YAP for organ size control.

### Example 3—To Test the Hypothesis that iNPCo-EVs Promote In Situ Neural Differentiation and Tissue Regeneration in an Ischemic Stroke Model

In this example, iNPCo-EVs were labeled using nanoparticles for noninvasive imaging in vivo. Based on an analysis of embryonic-like ECMs secreted by stem cells during neural development, this example focused on the heparan sulfate proteoglycans, heparin (analog of heparan sulfate), and hyaluronic acid for promoting brain tissue development. Therefore, Hep-HA hydrogels were used to encapsulate iNPCo-EVs for injection. iNPCo-EVs were transplanted in rats after distal middle cerebral artery occlusion. The animals were monitored by non-invasive magnetic resonance imaging to study neural regeneration and EV biodistribution. Behavior improvement was monitored for two months. Histological assessments were performed to reveal brain tissue regeneration.

Background and Rationale: Transplantations of NSC-EVs into rodent and porcine stroke models have been reported in a couple of studies. NSC-EVs promoted macrophage polarization toward an anti-inflammatory phenotype and increased the regulatory T-cell population in vivo after thromboembolic stroke. NSC-EVs also reduced lesion volume (based on T2-weighted sequences) and improved behavioral outcomes (e.g., coordination on balance beam) in aged mice. The mechanisms include anti-oxidative, pro-angiogenic, immunomodulatory, and neural plasticity regulating processes. In order to track EVs in vivo, EVs can be labeled with nanoparticles (4-6 nm).

However, for hPSC-NPCs transplanted into the cortex of rats after distal middle cerebral artery occlusion (MCAO), more complicated functions have not yet been restored. In addition, brain tissue regeneration and maturation are yet to be improved.

This example uses Hep-HA hydrogels to promote the EV retention and neural function.

HA is a major type of ECMs in brain and regulates synaptic function. HA hydrogels are suitable for brain tissue engineering and conjugation with heparin should promote growth factor-ECM binding. With the growing interests in using iPSC-EVs for stroke therapy, enhancing neural regeneration in vivo through EV encapsulation in HA hydrogels is important.

### Data

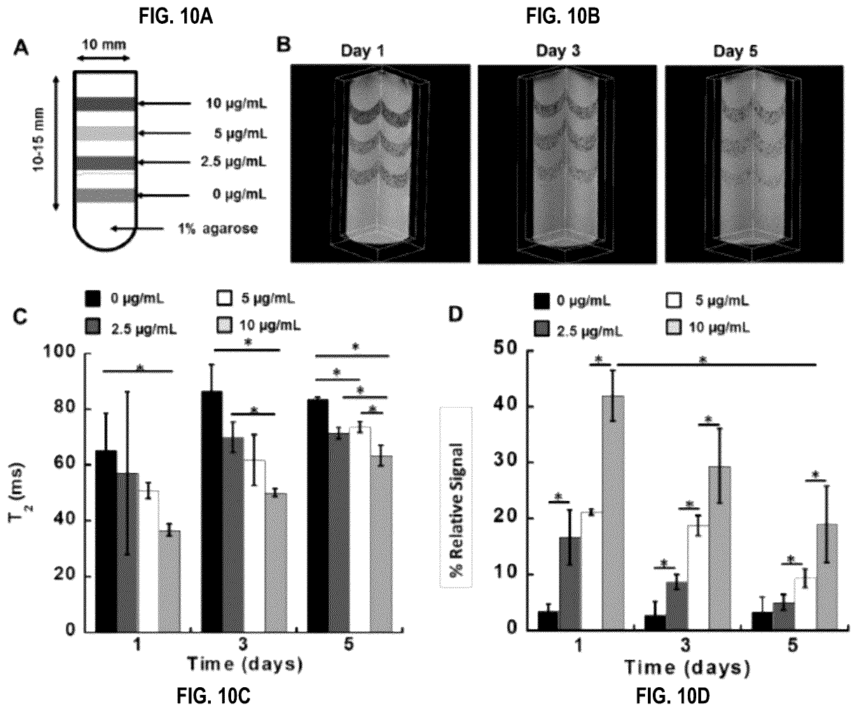

(a) NPC labeling with super-paramagnetic iron oxides (MPIO) for MRI tracking: NPCs were differentiated from mouse ESCs with high expression of Nestin and Musashi-1 (6080%). NPCs as 3-D aggregates were labeled with flash red fluorescent MPIO (Bangs Lab, part number ME03F/9772, 0.50-0.99 μm with the average size of 0.86 μm) with 70-80% efficiency. MRI tracking showed distinct signals from MPIO-labeled NPCs (FIG. 10).

Gradient recalled echo images showed a concentration-dependent reduction of T2 relaxation and T2* signal contrast. Quantification of T2 showed an increase of relaxation times over the entire time period, which was due to MPIO dilution resulting from cell division. These data demonstrate the feasibility of using MRI to track nanoparticle-labeled EVs.

(b) EV labeling with nanoscale iron oxides for non-invasive MRI tracking: Nanoscale iron oxides (8, 15, and 30 nm) were incubated with day 30 replated cortical spheroids for 48-72 hrs. Cells showed similar viability and metabolic activity compared to unlabeled cells. The iron oxides stimulated EV secretion. Additional modifications on iron oxide treatment can be made. Direct EV labeling with nanoparticles was also investigated using sonication and the EVs contained iron oxides. These data demonstrate the feasibility of using nanoscale iron oxides to label EVs for in vivo MRI tracking.

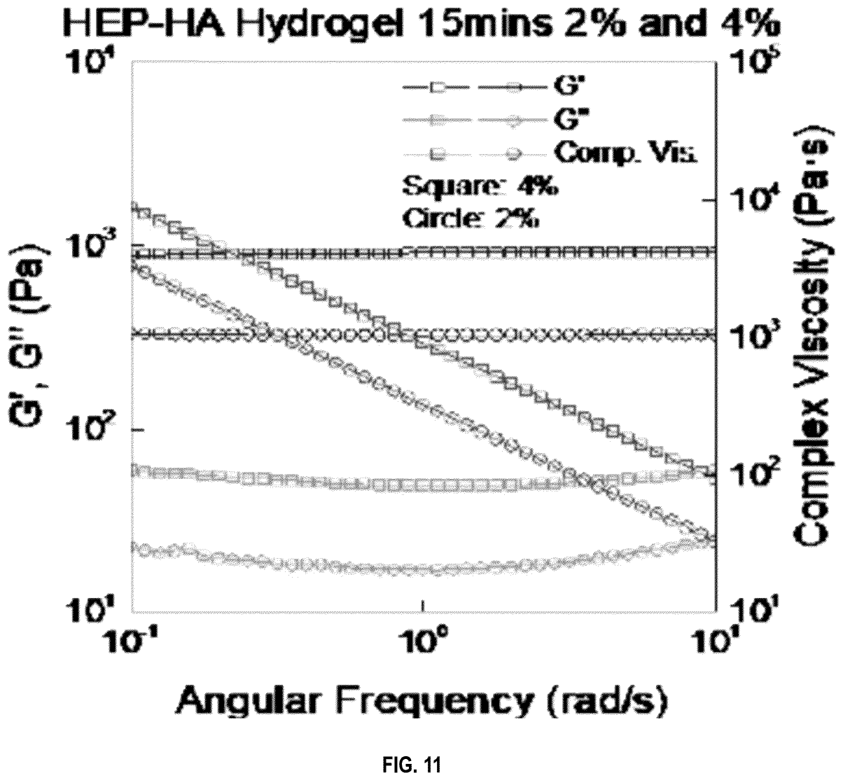

(c) Fabrication and characterization of Hep-HA hydrogels: Hep-HA hydrogels (with different modulus) prepared by photo-crosslinking of methylated HA and thiolated heparin (FIG. 11) have been fabricated and characterized.

Also evaluated were heparin-loaded HA hydrogels and the results indicate that heparin affects neural patterning of hiPSCs possibly by activating Wnt signaling. These preliminary data demonstrate the feasibility of using Hep-HA hydrogels to encapsulate the derived EVs.

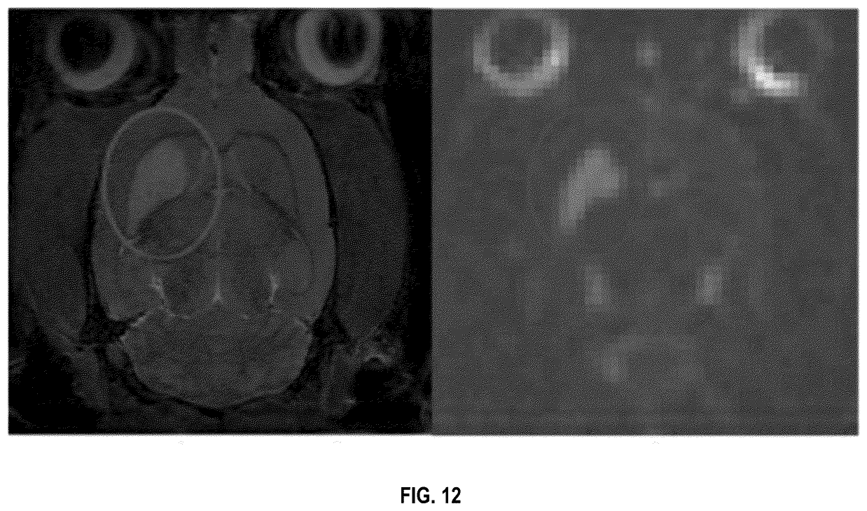

(d) In vivo MRI in an ischemic stroke rat model: Sodium (²³Na) MRI has been established as a reliable and quantifiable method for measuring the volume of ischemic lesion and subsequent tissue recovery. Utilizing a well-supported MCAO rat model of ischemic stroke with adaptations, it has been demonstrated that hypoxic hMSCs increased tissue recovery using ²³Na MRI (FIG.12), i.e., ²³Na-based volumetrics, as well as behavioral characterization, with correlations to immunohistochemistry assays of neuronal regeneration and astrocytic growth. These preliminary data demonstrate the feasibility of investigating MCAO rat models of ischemic stroke and using MRI for in vivo imaging.

### Methods

1. Nanoparticle labeling of EVs: For MRI monitoring, the EVs were labeled with nanoscale iron oxides (5-20 nm, from Sigma or gold nanoparticles from Luna Nanotech) for in vivo tracking. MPIO labeling has minimal adverse effects on neural cell physiology, and similar results were observed with iron oxides for human iPSC-NPCs. The EVs were incubated with iron oxides (50 μg/mL) overnight. Labeling efficiency was determined by flow cytometry.

The effect of EV type on intracellular uptake was evaluated by in vitro MRI monitoring. Specifically, an 11.75-T (500-MHz) vertical magnet (Magnex Scientific) with an 89-mm widebore equipped with a Bruker Avance console and Micro2.5 gradients was utilized to acquire images from all samples. Viability and proliferation of cells that uptake iron oxide-labeled EVs were compared with cells that uptake non-labeled EVs.

2. EV encapsulation in Hep-HA hydrogels: EV-containing hydrogels were created via gelation of Hep-HA under light for photo-crosslinking as shown in previous study. The patches were immersed in phosphate buffered saline at 37° C. EV release was quantified by NanoSight over 14-21 days. For EV release kinetics in vivo, laser light sheet microscopy was used to image explanted EV patches, day 0, 4, 7 post implantation. For in vivo EV uptake, EVs were labeled with PKH-67 or PKH-26 (Sigma) before they were encapsulated within hydrogel and implanted for histological analysis.

3. MCAO stroke rat model and EV transplantation: All procedures in this animal study were approved by IACUC. Both male and female were evaluated to determine if gender or corresponding sex hormones have a co-adjunctive impact. Briefly, Sprague-Dawley rats were subjected to 30 min of intraluminal filament MCAO as shown in previous publications. A 3.0-cm filament with a 0.35-mm thick and 3-4-mm long rubber coating (Doccol Corp, Sharon MA) was inserted through the ECA and guided 1.9 cm into the ICA until the MCA was blocked. Transient occlusion occurred for 1 h followed by removal of the filament, ECA ligation and wound suturing.

Intra-arterial delivery: A direct injection into the ICA was used with a micro-needle following modified procedures. This technique was chosen for promoting direct delivery to the brain while minimizing the risk for micro-emboli without significance disruption of blood flow.

Animals were randomly divided into six groups (n=12 per group) and receive transplanted EVs (low immunogenicity). Each animal as injected with a total of 108 EVs/g body weight according to the following groups: Group I: iPSC-EVs; Group II: iNPC-EVs (2-D); Group III: iNPCfo-EVs (forebrain organoids); Group IV: iNPCco-EVs (hindbrain cerebellum organoids); Group V: hMSC-EVs (a well-studied EV group); Group VI: vehicle only. A 5 μL of EV suspension in Hep-HA hydrogel at 1×1010 EVs/μL was injected. After 2 days, the rats were subjected for MRI tracking and behavioral tests. Intra-cerebral injection also was tested with EVs in hydrogels of low modulus to the injury site.

4. Non-invasive MRI tracking: Non-invasive imaging is crucial to assess the distribution of lesion, track the EVs, and monitor the cell survival, migration, function, and transplantation effect on the patient brain. Starting day 2 after transplantation and up to 2 months, MRI was performed on a 21.1T, 900-MHz vertical magnet built at the National High Magnetic Field Laboratory, equipped with a Bruker Avance spectrometer and Micro2.5 imaging gradients (1 T/m maximum strength). Animals were anesthetized with isoflurane, and the respiratory and heartbeats monitored at a controlled the temperature of 37° C. MRI parameters were optimized for this experiment. High-resolution three-dimensional GRE images were acquired and analyzed using Bruker ParaVision 3.1.2. Transplantation volumes were calculated for comparison of different groups.

5. Behavioral testing: Behavioral evaluation was performed using Montoya's staircase test. The animals were trained prior to MCAO surgery and the same procedure was followed during the tests after the transplantation. The animals were food-deprived before entering the staircase apparatus (Campden Instruments Ltd). Two food pellets were placed on each step of two staircases located one on either side of a central platform. The animals can reach down either side of the platform to grasp, lift, and retrieve food pellets.

The number of the pellets removed provided a quantifiable measure of the distance and efficiency of reaching skills.

6. Immunohistochemistry and in vivo trophic effects: After one-week or one-month follow-up, the animals were sacrificed by transcardial perfusion with phosphate-buffered saline followed by 4% paraformaldehyde. The brains were cryoprotected in an increasing gradient of 10, 20, 30% sucrose solution and cryostat sections at 40 μm were processed for immunostaining. Adjacent sections were stained for the regenerated neural cells and their differentiation status using antibodies for markers of NPC (Nestin, Sox2) and neurons (MAP2, Tuj1), a marker of astrocyte (GFAP) and oligodendrocyte (CC1), and a marker of cell proliferation (Ki67). To evaluate in vivo trophic effects, the tissues from the ischemic area were collected. Expression of matrix-bound trophic factors FGF-2, BDNF, and VEGF in the brain tissue are assessed by Western blot and 20 RT-PCR.

7. Functional tissue regeneration: To assess neuronal activity and synaptic connectivity at the injured site, electrophysiological and Ca2+ imaging analyses were performed. Spontaneous electrical activities (i.e., spiking) of tissue slice were examined by whole-cell recording at a current-clamp configuration. Neuronal activity at a circuit level was assessed by measuring field potentials (extracellular recording), postsynaptic potentials (whole-cell recording of single neurons), and Ca²⁺ spikes in Ca²⁺ indicator (e.g., Fluo-4AM) loaded neurons. The dependency of these signals on synaptic connections was determined by evaluating the changes induced by tetrodotoxin which blocks action potential, or by 6-cyano-7-nitroquinoxaline-2,3-dione (CNQX) which inhibits the major excitatory post-synaptic receptor, AMPAR.

Expected outcomes: Transplantation of iNPCo-EVs may improve the tissue repair, neuron maturation, and functional recovery after stroke; (2) the contribution of the trophic function for tissue regeneration is delineated; (3) the embryonic neural tissue development and EV biodistribution in vivo on the site of injury may be demonstrated using iNPCo-EVs through the MRI tracking.

### Alternative Strategies

For indirect EV labeling, cargo analysis can be required to elucidate the effects of iron oxides on the expression of therapeutic molecules in EVs from brain organoids. It is possible to use extrusion methods to isolate nano-vesicles containing iron oxides.

In addition, EV labeling using different nanoparticles (e.g., gold nanoparticles) was investigated as an alternative. Implantation of iNPCo-EVs with a defined number requires accurate control of the size and modulus of the hydrogels. The hydrogel patch size (5-7 mm in diameter) may be optimized.

For in vivo trophic effects, the expression of trophic factors FGF-2, BDNF, and VEGF was analyzed for their effects on cell migration, neurogenesis, and angiogenesis. The contribution of other trophic factors was examined through proteomics analysis.

Statistical rigor: Sample size as determined from estimates of expected mean and variance derived from data generated or obtained from the literature. A minimum of 3 biological and 3 technical replicates were performed, and post hoc power analyses were used to confirm appropriate p-value. Differences in cell and functional data were classified via t-tests and ANOVA, and transcriptomic data via Principal Component Analysis. Data normality and homoscedasticity were determined using Shapiro-Wilkes and Levene's test; outliers were identified via Dixon's Q-test (n<6) or Grubbs' test (n>6). For studies that violate homoscedasticity/normality, nonparametric (two-sample Mann-Whitney; Kruskal-Wallis) tests were used. If the ANOVA shows significant differences, post hoc pairwise comparison via Tukey's HSD was performed.

That which is claimed is:

1. A method comprising:
producing extracellular vesicles (EVs) from human induced pluripotent stem cells (hiPSCs) by
forming 3-D brain organoids of the hiPSCs in a dynamic bioreactor; and
collecting the EVs from the bioreactor.

2. The method of claim 1, wherein the dynamic bioreactor is at least one of a wave motion bioreactor, a suspension bioreactor, and a vertical wheel bioreactor.

3. The method of claim 1, further comprising encapsulating the collected EVs in a cross-linked hydrogel.

4. The method of claim 3, wherein the cross-linked hydrogel is a heparin-hyaluronic acid hydrogel.

5. The method of claim 1, wherein the brain organoids are forebrain cortical organoids and/or hindbrain cerebellar organoids.

6. The method of claim 1, wherein forming 3-D brain organoids of the hiPSCs in a dynamic bioreactor includes providing neural spheroids in a hydrogel and inoculating the neural spheroids in the presence of cyclopamine and FGF-2.

7. The method of claim 1, wherein the EVs include miRNA that contributes to neurogenesis.

8. The method of claim 1, wherein the EVs promote recovery after an ischemic injury.

9. A product comprising a pharmaceutical dosage form comprising extracellular vesicles (EVs) of brain organoids from human induced pluripotent stem cells (hiPSCs).

10. The product of claim 9, wherein the pharmaceutical dosage form includes the EVs in a cross-linked hydrogel.

11. The product of claim 10, wherein the cross-linked hydrogel is a heparin-hyaluronic acid hydrogel.

12. The product of claim 9, wherein the brain organoids are forebrain cortical organoids and/or hindbrain cerebellar organoids.

13. The product of claim 9, wherein the EVs include miRNA that contributes to neurogenesis.

14. The product of claim 9, wherein the EVs promote recovery after an ischemic injury.

15. The product of claim 9, wherein the pharmaceutical dosage form is configured to inject the EVs into a patient.

16. A method of treatment comprising administering to a patient in need thereof a pharmaceutical dosage form comprising extracellular vesicles (EVs) of brain organoids from human induced pluripotent stem cells (hiPSCs).

17. The method of claim 16, wherein the patient has an ischemic condition.

18. The method of claim 16, wherein the pharmaceutical dosage form includes the EVs in a cross-linked hydrogel.

19. The method of claim 18, wherein the cross-linked hydrogel is a heparin-hyaluronic acid hydrogel.

20. The method of claim 16, wherein the brain organoids are forebrain cortical organoids and/or hindbrain cerebellar organoids.

21. The method of claim 16, wherein the EVs include miRNA that contributes to neurogenesis.

22. The method of claim 16, wherein the EVs promote recovery after an ischemic injury.

23. The method of claim 16, wherein administering the pharmaceutical dosage form includes injecting the pharmaceutical dosage form into the patient.
